(19) United States
(12) Patent Application Publication
(10) Pub. No.: US 2015/0223956 A1
(43) Pub. Date: Aug. 13, 2015

(54) ANCHORED NON-SPHERICAL BALLOON FOR THE TREATMENT OF OBESITY

(71) Applicant: Children's National Medical Center, Washington, DC (US)

(72) Inventors: Evan P. Nadler, Washington, DC (US); Kevin Cleary, Potomac, MD (US); Anthony Sandler, Bethesda, MD (US); Miller Hamrick, Silver Spring, MD (US)

(73) Assignee: Children's National Medical Center, Washington, DC (US)

(21) Appl. No.: 14/621,075

(22) Filed: Feb. 12, 2015

Related U.S. Application Data
(60) Provisional application No. 61/938,958, filed on Feb. 12, 2014.

(51) Int. Cl. A61F 5/00; A61B 1/313; A61B 1/273

(52) U.S. Cl. CPC A61F 5/003; A61F 5/0036; A61F 5/0089; A61B 1/2736; A61B 1/3132

(57) ABSTRACT

Described herein are a device and an associated method for treatment of obesity. Specifically, a non-spherical balloon is described that expands inside the stomach to reduce luminal volume. The non-spherical balloon is firmly anchored to the abdominal wall in order to eliminate migration risks. Further, the device inflates in a longitudinal fashion to fill a greater curvature of the stomach, thereby preventing food from contacting the ghrelin-producing cells that stimulate hunger.

### INCORPORATION BY REFERENCE

This disclosure claims the benefit of U.S. Provisional Application No. 61/938,958, filed on Feb. 12, 2014, the disclosure of which is incorporated herein by reference in its entirety.

### FIELD OF INVENTION

The present disclosure relates to a device and an associated method of using the device for the treatment of obesity. Specifically, the present disclosure is directed to a non-spherical balloon that is anchored in the gastric cavity for treating obesity.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Obesity has become the world's most pressing healthcare issue and severe obesity affects nearly four percent or 2.7 million children. According to a recent survey, the obesity epidemic in the United States affects approximately 15% of the pediatric population. In adolescents alone, it is estimated that over 1 million children have a body-mass index (BMI) greater than 35 kg/m², which is the threshold for weight loss surgery in the adult population. Obesity is associated with a myriad of physical and psycho-social conditions that negatively impact patients' health and quality of life. While lifestyle modification and patient education are important components of any weight management program, they alone have failed to demonstrate promising and consistent weight loss results.

Bariatric surgery procedures have gained some acceptance for adolescents with morbid obesity, as the likely candidate to provide significant and sustainable weight loss results. However, according to nationally conducted surveys the number of bariatric surgeries in adolescents has plateaued despite the increasing number of patients who would qualify for such procedures. For instance, in adolescents, only approximately 1,000 bariatric procedures are performed per year, which makes up approximately 0.1% of patients that would benefit from the surgery. The reasons behind the aversion to weight loss surgery are not clear, but contributing factors may include a reluctance to undergo a non-reversible surgical procedure that alters the intestinal anatomy (Roux-en-Y gastric bypass) or permanently shrinks the stomach size (sleeve gastrectomy), or a general fear of the risks associated with any surgical procedure. Additionally, patients are averse to participate in such bariatric surgeries due to considerable insurance hurdles associated with these invasive procedures.

In an effort to provide less invasive and potentially temporary solutions, several organizations have attempted to develop devices to reduce stomach volume or absorptive capacity, but these devices have met with very limited success. For instance, the laparoscopic adjustable gastric band is currently the only Food and Drug Administration (FDA) approved device. However, it makes up only 4% of bariatric procedures performed in the US due to its high complication rate and the requirement to remove the device from the patient after a few years. Endoscopic techniques have also been explored, including intra-gastric balloons and barrier devices. Although several of these devices are available outside of the US, they are not yet approved by the FDA in large part due to the possibility for migration of the device in the gastrointestinal tract. Furthermore, the intra-gastric balloon technique is typically not recommended for use greater than six months due to the risk of balloon rapture, which may cause intestinal obstruction and eventual death of the patient. Additionally, intra-gastric balloons have been hampered by functional issues related to their design, such as difficulty in changing balloon volume once implanted, inability to determine the patient's optimum balloon volume and the like. Accordingly, such surgical procedures are seldom, if at all, recommended for adolescent children.

Furthermore, despite the fact that volumes of published literature have demonstrated that behavioral, medical, and lifestyle treatments do not work for majority of patients, younger children who suffer from obesity related illnesses are less likely to be referred to such invasive surgery procedures due to the associated risks. Accordingly, there is a requirement to develop a less invasive and effective therapy that overcomes the risks associated with bariatric surgical procedures and that appeals to patients (especially children) who suffer from obesity related illnesses.

### SUMMARY

The present disclosure provides for a weight loss device and an associated method thereof for using the device that completely eliminates the risk of device migration. Furthermore, the device provides for an additional mechanism for weight loss other than volume reduction. Specifically, according to an embodiment of the present disclosure there is provided a non-spherical balloon that is firmly anchored to the abdominal wall in order to eliminate device migration risks. The balloon expands in a longitudinal manner inside the stomach to reduce the luminal volume and fills the greater curvature of the stomach, thereby preventing food from contacting the ghrelin producing cells (found in the greater curvature of the stomach) that stimulate hunger.

According to one embodiment, the non-spherical balloon can be anchored in the abdominal cavity of the patient in a fashion similar to the technique of inserting a gastrostomy tube button. Specifically, the non-spherical balloon can be laparoscopically delivered and used for weight loss applications. Since gastrostomy tubes with external filling ports are used for feeding children, anchoring the non-spherical balloon in such a manner to treat obesity would gain high acceptance by children, their parents, and their physicians. Furthermore, having an external port for the non-spherical balloon provisions for an easy mechanism to inflate and deflate the balloon based on individual patients anatomy.

The foregoing paragraphs have been provided by way of general introduction, and are not intended to limit the scope of the following claims. The described embodiments together, with further advantages, will be best understood by reference to the following detailed description taken in conjunction with the accompanying drawings.

### BRIEF DESCRIPTION OF THE DRAWINGS

Various embodiments of this disclosure that are provided as examples will be described in detail with reference to the following figures, wherein like numerals reference like elements, and wherein:

FIG. 1 depicts according to an embodiment, a schematic of a stomach of a human body;

FIG. 2A depicts an exemplary geometrical representation of a non-spherical balloon, FIG. 2B depicts a frontal-top view of the non-spherical balloon, and FIG. 2C depicts a non-spherical balloon assembly;

FIG. 3 illustrates a comparison of implanting a non-spherical balloon according to one embodiment to a sleeve gastrectomy procedure; and

FIG. 4 illustrates a block diagram of a computing device according to one embodiment.

### DETAILED DESCRIPTION OF EMBODIMENTS

Obesity has been established as a critical health issue. The consequences of morbid obesity include an increased risk of cardio-vascular disease (especially hypertension), dyslipidemia, diabetes mellitus, gallbladder disease, increased prevalence and mortality of selected types of cancer, and socioeconomic and psychosocial dysfunction. Obesity in the pediatric population has reached epidemic proportions, with nearly one in three children being overweight or obese. The problem has reached a point where diseases such as type II diabetes, previously not seen in children, are now are increasingly being diagnosed.

The onset of obesity in a patient is diagnosed by computing a parameter referred to herein as body-mass index (BMI). The BMI of a person is calculated based on the person's weight and height. BMI provides a reliable indicator of body thinness or thickness and is used to screen for weight categories that may lead to health problems. Further, BMI for children in the ages from 2 years to 20 years is used in a slightly different manner for diagnosing purposes. Specifically, the BMI is calculated in the same way as for adults, but is further compared to typical BMI values for other children of the same age. In other words, instead of performing a comparison against fixed thresholds for underweight and overweight, the BMI is compared against a percentile for children of the same gender and age. A BMI that is less than the 5th percentile is considered underweight and above the 95th percentile is considered obese. Children with a BMI between the 85th and 95th percentile are considered to be overweight.

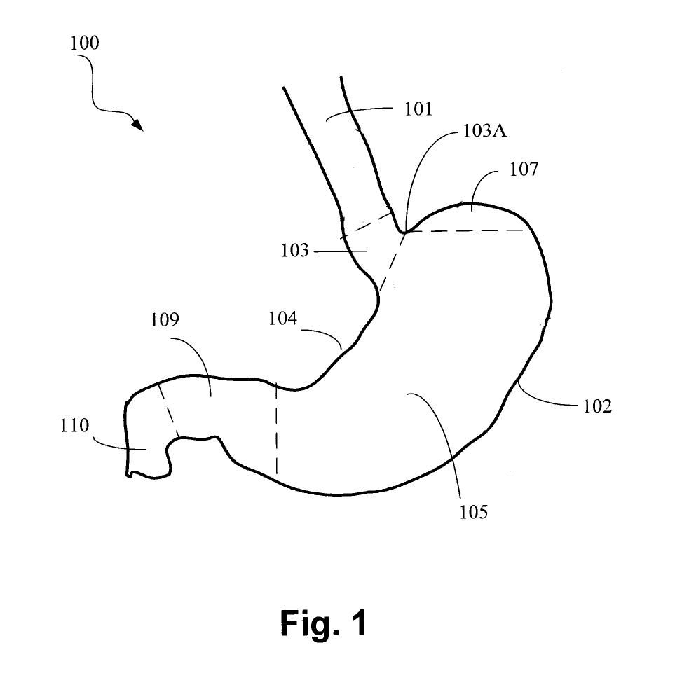

Turning now to FIG. 1 which illustrates a schematic diagram of a stomach 100 of a human body. The stomach lies between the esophagus 101 and the duodenum (the first part of the small intestine) 110. The stomach has a ‘J’ shape and is located in the upper-left area of the abdomen below the liver and next to the spleen. The stomach's main component for digestion is a powerful mix of secretions collectively referred to herein as gastric juices. To counteract these strong juices, the stomach protects itself with mucus-like secretions.

The stomach 100 is divided into four sections that include cardia 103, body 105, fundus 107 and Pylorus 109. Each of these portions includes different cells that perform different functions. The cardia 103 is the portion of the stomach where the contents of the esophagus empty into the stomach body 105. The cardia 103 includes a muscular ring referred to as an esophageal sphincter (esophageal valve) that is disposed near the cardia notch 103A. As food reaches the end of the esophagus 101, the food enters the stomach body 105 through the esophageal valve.

The body of the stomach 105 is the main central region of the stomach. The body is defined by a posterior and anterior curvatures of the stomach referred to herein as the lesser curvature 104 and greater curvature 102. The greater curvature 102 of the stomach is approximately four to five times as long as the lesser curvature 104. The fundus 107 is the portion of the stomach that is formed by the upper curvature of the stomach. The Pylorus 109 is the lower portion of the stomach that facilitates emptying the contents into the small intestine via the duodenum 110. Specifically, the pylorus 109 includes a pyloric sphincter (muscular valve) that enables food to pass from the stomach body 105 to the small intestine.

According to one embodiment of the present disclosure, a non-spherical balloon is anchored to the abdominal wall (along the greater curvature) in order to eliminate migration risk of the balloon. The balloon is positioned in a manner such that the balloon can inflate in a longitudinal manner in order to fill the greater curvature 102 of the stomach. Ghrelin-producing cells found along the greater curvature 102 of the stomach are known to stimulate hunger and subsequent food intake. Thus, by inflating the balloon in the longitudinal manner, there is provided the advantageous ability of preventing food from contacting the ghrelin-producing cells and thereby addresses the obesity problem.

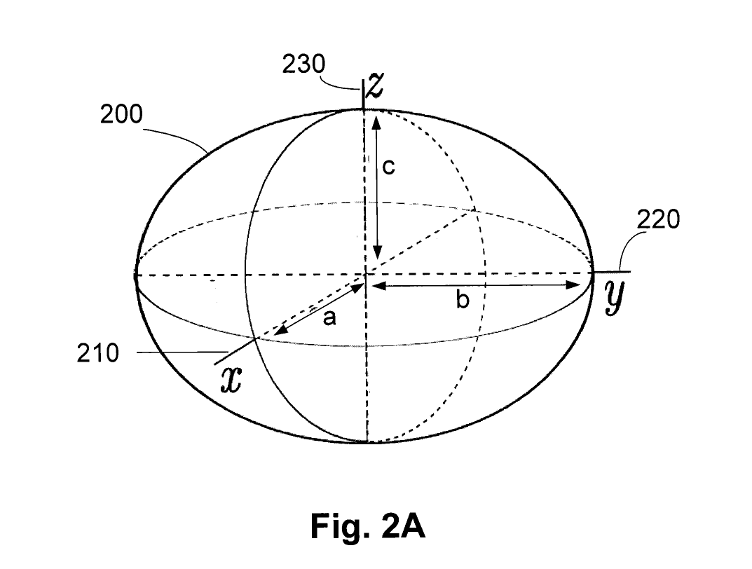

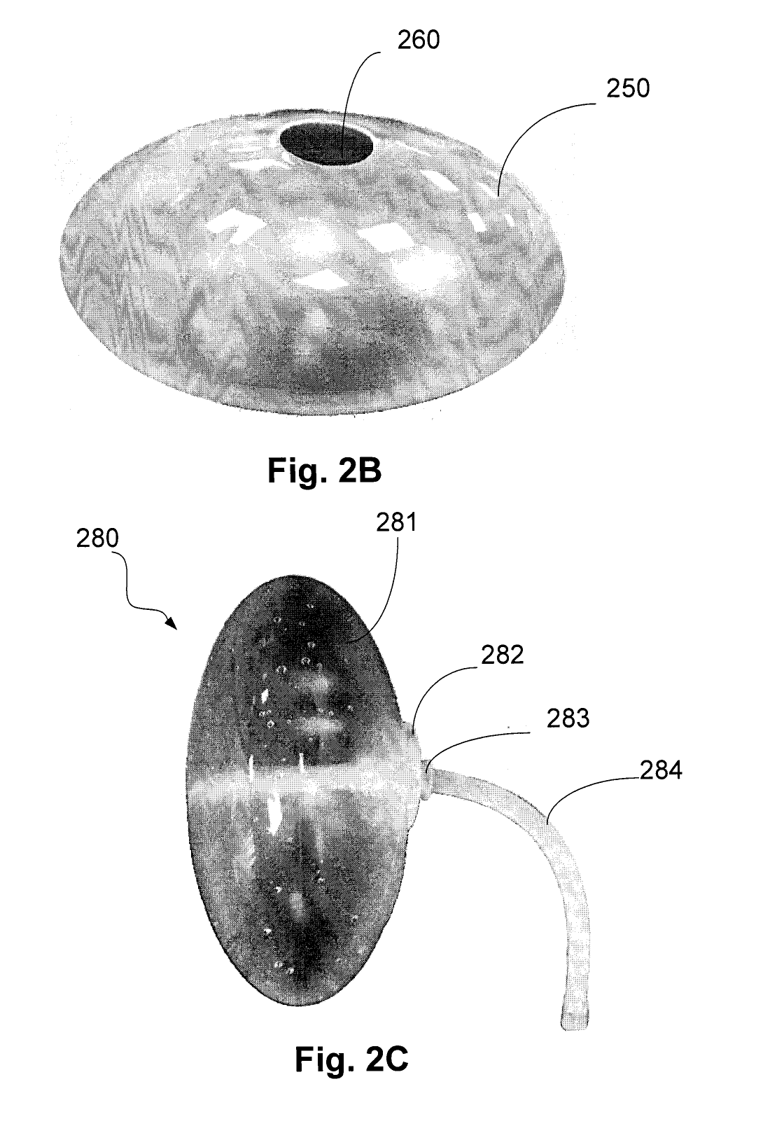

FIG. 2A depicts an exemplary geometrical representation of a non-spherical balloon 200. The non-spherical balloon 200 has an ellipsoidal like shape and is defined in a cartesian co-ordinate system based on three axes: the x-axis 201 referred to herein as a latitudinal axis, the y-axis 202 referred to herein as a longitudinal axis, and a z-axis 203 referred to herein as a vertical axis. As depicted in FIG. 2A, the half lengths of the axes of the non-spherical balloon are represented by parameters ‘a’, ‘b’ and ‘c’ for the x, y, and z axes, respectively.

According to one embodiment, the non-spherical balloon can be represented by the following equation:

$$\frac{{x}^{2}}{{a}^{2}} + \frac{{y}^{2}}{{b}^{2}} + \frac{{z}^{2}}{{c}^{2}} = 1 \qquad ( 1 )$$

The non-spherical balloon may be one of a scalene ellipsoid (a>b>c), an oblate ellipsoid (a=b>c), and a prolate ellipsoid (a=b<c). However, the balloon is constrained from having a spherical shape (a=b=c). Therefore, by manufacturing the balloon to have an ellipsoidal like shape provides the advantageous ability of anchoring the balloon at a point that lies along the greater curvature of the stomach and further inflating the balloon in a longitudinal manner in order to fill the greater curvature of the stomach. In doing so, food is prevented from being in contact with the ghrelin-producing cells that lie in the greater curvature of the stomach.

FIG. 2B depicts, according to an embodiment, a frontal-top view of a non-spherical balloon 250. According to one embodiment, the non-spherical balloon 250 is made of a plastic polymer or soft, well-tolerated silicone material. The non-spherical balloon is biocompatible in accordance with ISO 10993-1 2009 standards. Furthermore, the balloon can be subjected to several forms of testing to ensure that the balloon does not experience any significant mechanical or chemical degradation. For instance, the non-spherical balloon 250 can be subjected to testings that include cytotoxicity testing, murine local lymph node testing using aqueous and non-aqueous methodology, intra-cutaneous testing according to the International Standards Organization (ISO) using aqueous and non-aqueous methodology, systemic toxicity testing according to the ISO and the United States Pharmacopeia, and the like. Furthermore, the non-spherical balloon 250 includes an aperture denoted by 260 that is anchored to the stomach (within the abdominal wall) and provides an aperture to fill the balloon with water in order to expand the balloon in a longitudinal manner along the greater curvature of the stomach. In alternative embodiments, air, saline or other substances can be used in addition to or in place of water.

FIG. 2C depicts, according to an embodiment, a non-spherical balloon assembly 280. The assembly includes a non-spherical balloon 281, an anchor 282, an inflation port 283, and an inflation tube 284. According to one embodiment, the balloon implant can be fit through and delivered to the patients' abdomen by using minimally invasive procedures (described below in detail). Such procedures employ, in one example, a trocar device (i.e., a medical device having a metallic/plastic sharpened or non-bladed tip and a hollow tube and seal that is used for abdominal surgeries) having an inner diameter of, for example, 12-15 millimeters to place the balloon. By employing an anchor 282, the balloon is held affixed in one position during normal digestive cycles, and is thus prevented from migrating in the stomach, for instance beyond the pylorus, thereby avoiding any serious medical conditions. The non-spherical balloon 281 is available in multiple sizes for different sized patients.

According to one embodiment, the anchor 282 can be a silicone sliding disc (for example, a percutaneous endoscopic gastrostomy (PEG) disc with a piece of tubing external to the body) or a button (such as Mic-key balloon button or mini one balloon button) that is firmly anchored within the abdominal wall and embedded under the patient's skin. The inflation port 283 provides an ingress point for the inflation tube 284 and is made easily accessible to inflate/deflate the balloon to a desired size. Alternatively, according to one embodiment, the inflation port may be embedded in the skin of the patient, thereby providing a visual access to inflate/deflate the balloon using the inflation tube. The inflation port may also be positioned under the skin in one alternative embodiment. The inflation port may be, in one embodiment, a luer activated inflation port. The inflation port may, in one embodiment, be a laparoscopic adjustable gastric band (lap-band) type port. In another embodiment, the lap-band type port may be positioned lateral to a trocar opening under the skin.

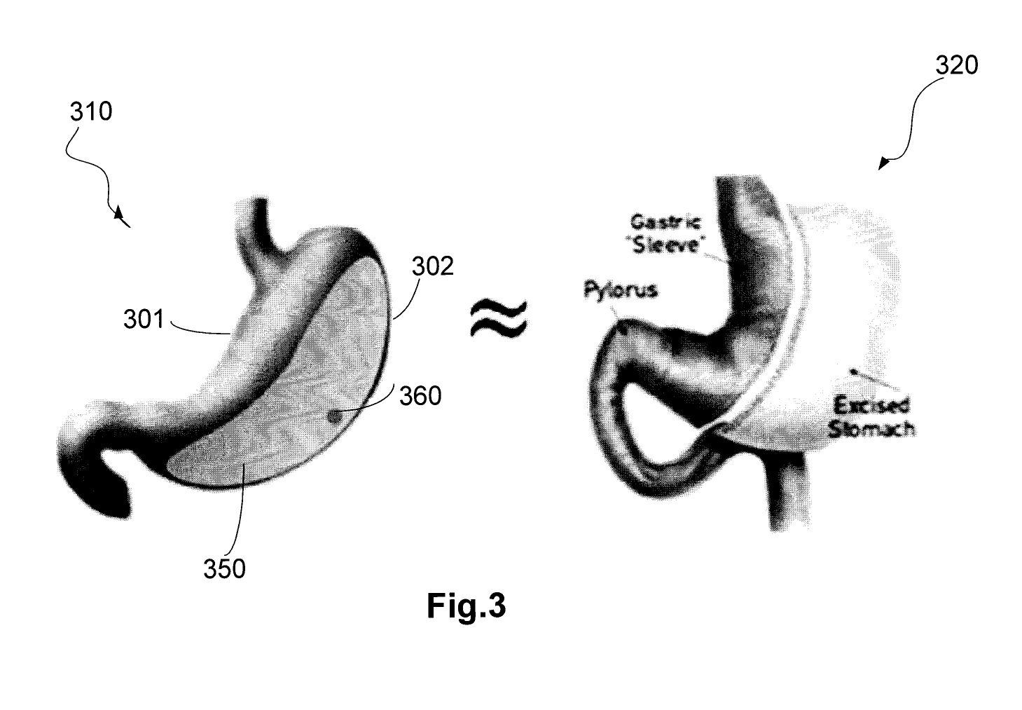

FIG. 3 illustrates, according to an embodiment, a comparison of implantation (represented by 310) of a non-spherical balloon to a sleeve gastrectomy procedure represented as 320. In the implantation of the non-spherical balloon, the balloon 350 having an aperture 360 is implanted in a manner such that the balloon expands in a longitudinal manner along the larger curvature 302 of the stomach. Thus, as described previously, the food taken in by a patient is avoided from contacting with the ghrelin-producing cells. Further, the volume of the stomach between the larger curvature 302 and the lesser curvature 301 is reduced and thus the balloon provides a feeling of satiety to the patient.

In contrast, the sleeve gastrectomy procedure 320 is a surgical weight-loss procedure in which the stomach is reduced to about 25% of its original size, by surgical removal of a large portion of the stomach along the greater curvature resulting in a sleeve or tube like structure. The procedure permanently reduces the size of the stomach, although there could be some dilatation of the stomach later on in life. Thus, the technique of implanting the non-spherical balloon according to the present disclosure achieves a reduction in volume of the stomach (and thereby provides a solution for obesity) without having to perform invasive surgical procedures such as those associated with the sleeve gastrectomy process.

In what follows a description of techniques of placing the non-spherical balloon are provided. According to one embodiment, the non-spherical balloon can be placed in the stomach using an endoscopic approach as used with gastrostomy tubes. In such a process, an endoscope is passed into the mouth, down the esophagus, and into the stomach. The surgeon can then see the stomach wall through which the tube (such as a percutaneous endoscopic gastrostomy (PEG) tube) will pass. Under direct visualization with the endoscope, the tube passes through the skin of the abdomen, through a very small incision, and into the stomach. The non-spherical balloon can then be blown from an inflation port disposed at the end of the tube. It must be appreciated that, by this process, the balloon can be anchored in one position and held affixed to the stomach wall. Thus, potential migration problems associated with the balloon movements can be avoided. Furthermore, such procedures usually avoid the need for general anesthesia and a large incision.

Alternatively, the non-spherical balloon can be positioned in the stomach by using a laparoscopic technique. Laparoscopic or “minimally invasive” surgery is a specialized technique for performing surgery. In usual “open” surgery, the surgeon uses a single incision to enter into the abdomen. In contrast, laparoscopic surgery uses several 0.5 cm-1 cm sized incisions. Each incision is called a “port”. At each port, a tubular instrument (trocar) is inserted. Specialized instruments and a special camera known as a laparoscope are passed through the trocars during the procedure. At the beginning of the procedure, the abdomen is inflated with carbon dioxide gas to provide a working and viewing space for the surgeon. The laparoscope transmits images from the abdominal cavity to high-resolution video monitors. During the operation the surgeon can view detailed images of the abdomen on the monitor. This approach also allows the surgeon to perform the operations smaller incisions.

Alternatively, according to one embodiment, techniques such as CT-scanning, MRI, 3-dimensional imaging and the like can be employed to determine the morphological features of the stomach of the patient. Further, an incision point for inserting the balloon in the abdomen can also be determined from analyzing the CT-scans of the abdomen of the patient. For instance, an appropriate position for tube placement on the stomach can be identified as the position where the confluence of the gastroepiploic vessels occurs. Alternatively, the non-spherical balloon can be anchored at a midpoint along the larger curvature in order to provision for uniform expansion of the balloon. Thus, employing the above described techniques provides advantageous features such as: the balloon volume can be tailored for each pediatric patient and an amount of obstruction of the greater curvature of the stomach to impact the ghrelin production can be determined for each patient. According to one embodiment, the balloon is inflated to occupy approximately 70% of the stomach volume following implantation and expansion. This value has particularly positive results. Other percentages are also possible. In one alternative embodiment, the tailoring of each balloon can include custom manufacturing each balloon via 3D printing after obtaining the imaging study to determine stomach volume.

Furthermore it must be appreciated that the present disclosure provides techniques that incur benefits such as: reducing the risk of accidental deflation and migration as the gastrostomy tube has long been accepted as suitable for long-term use with an excellent safety profile, allowing a customized balloon volume to achieve a patient's feeling of fullness and satiation without blocking food flow and/or causing stretching or necrosis of the stomach wall, conforming to the greater curvature of the stomach and blocking its contact to food, which is believed to play a key signaling role which adds to the efficacy of bariatric surgery, and allowing the flexibility to cycle the balloon volume reducing the risk of ulcers and/or bleeding, and common treatment side effects such as vomiting and nausea.

Additionally, the operations of placement as well as inflating the balloon with water can be monitored and controlled or automatically operated by a processing by one or more processing circuits in an alternative embodiment which is in addition to the embodiment of manual placement and inflation. Manual inflation can also be utilized for both the fixed size balloon and the variable sized balloon. For example, operation of the inflation can be based on measured or predetermined triggers or time values. The inflation tube can be connected to water pump and the rate of water inflation can be monitored and controlled by a computer.

In addition, in another alternative embodiment, remote expansion of the balloon can be computer controlled/operated. In particular, in one application of this embodiment, the balloon is anchored from the inside and the balloon is inflated or deflated based on instructions from the processing circuitry/computer.

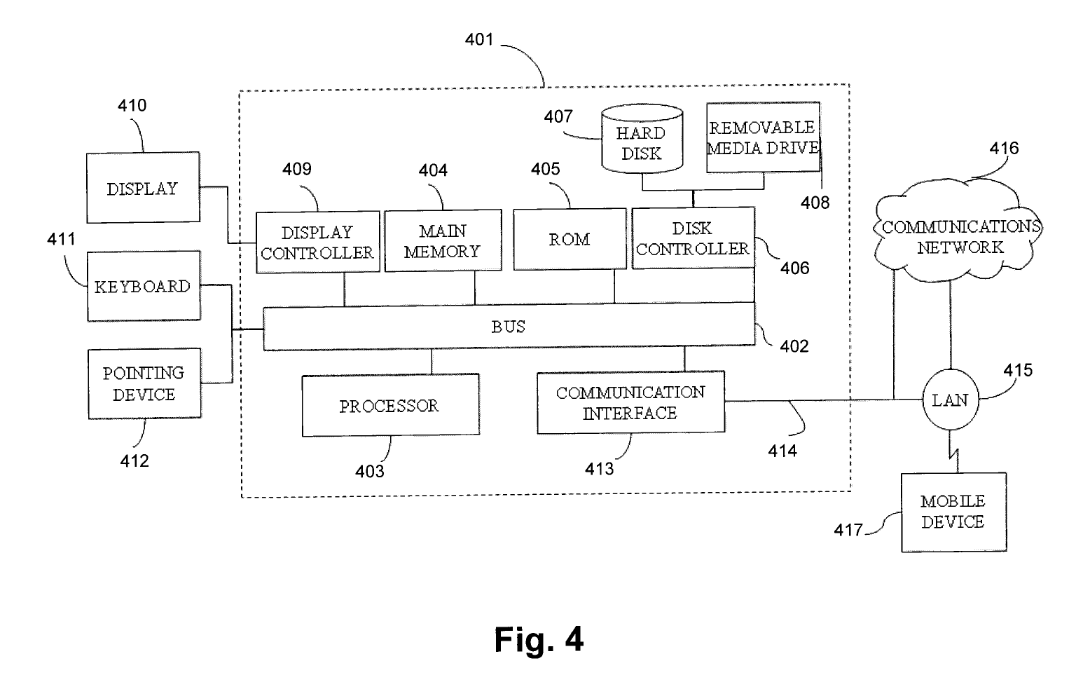

A processing circuit includes a programmed processor (for example, processor 403 in FIG. 4), as a processor includes circuitry. A processing circuit also includes devices such as an application-specific integrated circuit (ASIC) and conventional circuit components arranged to perform the recited functions. The various features discussed above may be implemented by a computer system (or programmable logic). FIG. 4 illustrates such a computer system 401.

The computer system 401 includes a disk controller 406 coupled to the bus 402 to control one or more storage devices for storing information and instructions, such as a magnetic hard disk 407, and a removable media drive 408 (e.g., floppy disk drive, read-only compact disc drive, read/write compact disc drive, compact disc jukebox, tape drive, and removable magneto-optical drive). The storage devices may be added to the computer system 401 using an appropriate device interface (e.g., small computer system interface (SCSI), integrated device electronics (IDE), enhanced-IDE (E-IDE), direct memory access (DMA), or ultra-DMA).

The computer system 401 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 401 may also include a display controller 409 coupled to the bus 402 to control a display 410, for displaying information to a computer user. The computer system includes input devices, such as a keyboard 411 and a pointing device 412, for interacting with a computer user and providing information to the processor 403. The pointing device 412, for example, may be a mouse, a trackball, a finger for a touch screen sensor, or a pointing stick for communicating direction information and command selections to the processor 403 and for controlling cursor movement on the display 410.

The processor 403 executes one or more sequences of one or more instructions contained in a memory, such as the main memory 404. Such instructions may be read into the main memory 404 from another computer readable medium, such as a hard disk 407 or a removable media drive 408. One or more processors in a multi-processing arrangement may also be employed to execute the sequences of instructions contained in main memory 404. In alternative embodiments, hard-wired circuitry may be used in place of or in combination with software instructions. Thus, embodiments are not limited to any specific combination of hardware circuitry and software.

As stated above, the computer system 401 includes at least one computer readable medium or memory for holding instructions programmed according to any of the teachings of the present disclosure and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes.

Stored on any one or on a combination of computer readable media, the present disclosure includes software for controlling the computer system 401, for driving a device or devices for implementing the invention, and for enabling the computer system 401 to interact with a human user. Such software may include, but is not limited to, device drivers, operating systems, and applications software. Such computer readable media further includes the computer program product of the present disclosure for performing all or a portion (if processing is distributed) of the processing performed in implementing any portion of the invention.

The computer code devices of the present embodiments may be any interpretable or executable code mechanism, including but not limited to scripts, interpretable programs, dynamic link libraries (DLLs), Java classes, and complete executable programs. Moreover, parts of the processing of the present embodiments may be distributed for better performance, reliability, and/or cost.

The term “computer readable medium” as used herein refers to any non-transitory medium that participates in providing instructions to the processor 403 for execution. A computer readable medium may take many forms, including but not limited to, non-volatile media or volatile media. Non-volatile media includes, for example, optical, magnetic disks, and magneto-optical disks, such as the hard disk 407 or the removable media drive 408. Volatile media includes dynamic memory, such as the main memory 404. Transmission media, on the contrary, includes coaxial cables, copper wire and fiber optics, including the wires that make up the bus 402. Transmission media also may also take the form of acoustic or light waves, such as those generated during radio wave and infrared data communications.

Various forms of computer readable media may be involved in carrying out one or more sequences of one or more instructions to processor 403 for execution. For example, the instructions may initially be carried on a magnetic disk of a remote computer. The remote computer can load the instructions for implementing all or a portion of the present disclosure remotely into a dynamic memory and send the instructions over a telephone line using a modem. A modem local to the computer system 401 may receive the data on the telephone line and place the data on the bus 402. The bus 402 carries the data to the main memory 404, from which the processor 403 retrieves and executes the instructions. The instructions received by the main memory 404 may optionally be stored on storage device 407 or 408 either before or after execution by processor 403.

The computer system 401 also includes a communication interface 413 coupled to the bus 402. The communication interface 413 provides a two-way data communication coupling to a network link 414 that is connected to, for example, a local area network (LAN) 415, or to another communications network 416 such as the Internet. For example, the communication interface 413 may be a network interface card to attach to any packet switched LAN. As another example, the communication interface 413 may be an integrated services digital network (ISDN) card. Wireless links may also be implemented. In any such implementation, the communication interface 413 sends and receives electrical, electromagnetic or optical signals that carry digital data streams representing various types of information.

The network link 414 typically provides data communication through one or more networks to other data devices. For example, the network link 414 may provide a connection to another computer through a local network 415 (e.g., a LAN) or through equipment operated by a service provider, which provides communication services through a communications network 416. The local network 414 and the communications network 416 use, for example, electrical, electromagnetic, or optical signals that carry digital data streams, and the associated physical layer (e.g., CAT 5 cable, coaxial cable, optical fiber, etc.). The signals through the various networks and the signals on the network link 414 and through the communication interface 413, which carry the digital data to and from the computer system 401 may be implemented in baseband signals, or carrier wave based signals.

The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 401 can transmit and receive data, including program code, through the network(s) 415 and 416, the network link 414 and the communication interface 413. Moreover, the network link 414 may provide a connection through a LAN 415 to a mobile device 417 such as a personal digital assistant (PDA) laptop computer, or cellular telephone.

While aspects of the present disclosure have been described in conjunction with the specific embodiments thereof that are proposed as examples, alternatives, modifications, and variations to the examples may be made.

It should be noted that, as used in the specification and the appended claims, the singular forms “a,” “an,” and “the” include plural referents unless the context clearly dictates otherwise.

What is claimed is:

1. A method of treating obesity, the method comprising:
determining a point of anchoring a non-spherical balloon in a stomach of a patient;
placing the non-spherical balloon at the determined point by using a camera and a tubular equipment; and
elongating the non-spherical balloon along a longitudinal direction within the stomach by inflating the non-spherical balloon with water.
